(19) United States
(12) Patent Application Publication
(10) Pub. No.: US 2017/0346119 A1
(43) Pub. Date: Nov. 30, 2017

(54) ENERGY STORAGE IN CLOSED LOOP SYSTEMS USING MICROBIAL CONVERSION OF CARBON DIOXIDE TO HYDROCARBON FUEL

(71) Applicant: The Board of Trustees of the Leland Stanford Junior University, Stanford, CA (US)

(72) Inventors: Alfred M. Spormann, Stanford, CA (US); Oliver Wahnschafft, Winchester, MA (US)

(21) Appl. No.: 15/603,935

(22) Filed: May 24, 2017

Related U.S. Application Data
(60) Provisional application No. 62/342,100, filed on May 26, 2016.

(51) Int. Cl. H01M 8/0612; F02C 6/18; C12M 1/107; F02C 3/22; C10L 3/08; C12M 1/00; H01M 8/04082; F02C 3/34

(52) U.S. Cl. CPC H01M 8/0625; H01M 8/04216; F02C 6/18; F02C 3/34; F02C 3/22; C10L 3/08; C12M 43/04; C12M 23/36; C12M 21/04; C12M 43/08; C10L 2290/26

(57) ABSTRACT

The invention integrates electromethanogenesis, which uses electric energy to produce fuel, with a method to capture and recycle the carbon dioxide generated when the fuel is used to release energy.

### CROSS REFERENCE

This application claims benefit of U.S. Provisional Patent Application No. 62/342,100, filed May 26, 2016, which applications are incorporated herein by reference in their entirety.

The challenge of developing novel technologies for electrical energy storage has been identified as critical in the effort of promoting the accelerated adoption of renewable energy technologies. A key issue in the use of many renewable energy sources, such as solar, and wind is that the energy sources can fluctuate in availability over short periods of time, e.g. daily changes in sunlight; or over longer periods of time.

Development of a closed loop storage system would allow greater utilization of renewable energy sources. As an alternative to established battery technologies, whose application on large scale is hampered by costs and environmental concerns, the present invention is a system cycling fuel and carbon dioxide as primary media in high efficiency conversion processes.

### PUBLICATIONS

He et al. (2016) Environ. Sci. Water Res. Technol. 2:186-195; Kim et al. (2016) Biores. Technol. 208:58-63; Tian et al. (2016) Chem. Eng. J. 292:308-314; Ye et al. (2016) Electrochim. Acta. 194:441-447; Zhang et al. (2015) Environ. Sci. Water Res. Technol. 2(2):235-406.

Zhang and Lior (2008) Energy 33:340-351; Tan et al. (2008) Catalysis Today 131:292-304.

Giddings et al. (2015) Frontiers in Microbiology 6, article 468. US Patent publications 2012/0288898; 2016/0017800; 2016/0097138; 2014/0004578; 2015/0259669.

### SUMMARY OF THE INVENTION

Compositions and methods are provided for generating and storing energy by electrically-driven microbial conversion of carbon dioxide to hydrocarbon fuel, which, in turn, can release electrical and thermal energy when oxidized back to carbon dioxide to be stored.

The invention integrates electromethanogenesis, which uses electric energy to produce fuel, in conjunction with a method to capture and recycle the carbon dioxide generated when the fuel is used to release energy.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. closed-loop storage of electrical energy via methane as intermediate.

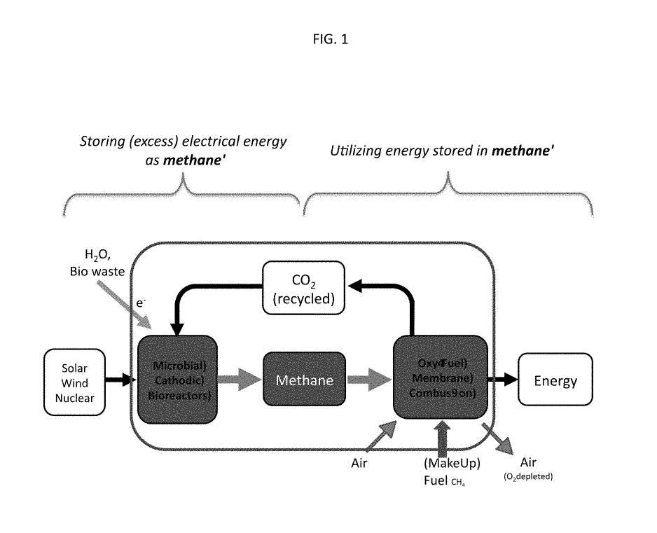

### DETAILED DESCRIPTION OF THE EMBODIMENTS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “an”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

An integrated system is provided for energy conversion and storage. The system comprises a first element of a microbial bioreactor that utilizes electric current to drive microbial conversion in a microbial fuel cell of CO₂ and water, or organic waste to hydrocarbons, including without limitation methane. A second element comprises a methane storage unit operably connected to the bioreactor and capable of storing methane produced by the bioreactor. The storage unit may comprise compressors, monitoring devices, out and input regulators and the like. A third element is operably connected to the methane storage unit, which third element converts methane to electricity or other forms of energy, such as thermal, or a combination thereof. In some embodiments this element is an oxyfuel combustion unit. In certain embodiments the CO₂ produced by combustion of the methane is recycled to the microbial bioreactor. The third element is generally linked to a transformer or other unit for transferring electric energy in a useful format.

The integrated system can be scaled as appropriate for the needs of the user, where, for example, a bioreactor can be operated at up to about 10 liter volume, up to about 50 liter volume, up to about 100 liter volume, up to about 500 liter volume, up to about 1000 liter volume, up to about 500 liter volume, up to about 10,000 liter volume, or more. The storage and combustion elements can be appropriately sized for the bioreactor.

In some embodiments, high efficiency electromethanogenesis uses electric energy to capture and recycle carbon dioxide. Various methods and devices can be used to provide the input electricity, for example photovoltaic cells, wind turbines, hydroelectric turbines and the like. In some embodiments the source of electricity is other than a conventional electric grid. In some embodiments an element for generation of electricity is included in the system of the invention.

Electromethanogenesis bioreactors comprise low redox potential electrons of a cathodic surface, operably linked by immersion to a bioreactor comprising methanogenic microorganisms, culture medium, and a means of capturing methane thus produced. An anodic surface is also present in the bioreactor, which is optionally but not necessarily immersed in the medium. The methanogenic microorganism may be a defined population including without limitation mixed species populations; a population selected for the desired methanogenic properties; a population of microbes combined with free extracellular enzymes that sorb to the electrode surface; and the like.

Microbial populations may comprise at least one, two, three or four families selected from the group consisting of Eubacteriaceae, Campylobacteraceae, Helicobacteraceae, Porphyromonadaceae, WCHB1-69, Spirochaetaceae, Deferribacteraceae, Rhodobacteraceae, Synergistaceae and Rhodocyclaceae. Thus, in some aspects, the microbial population mixture comprises bacteria from the Helicobacteraceae, WCHB1-69, Spirochaetaceae, and/or Synergistaceae families. In some specific aspects the mixture comprises bacteria from the genus Acetobacterium, Sulfurospirillum, Wolinella, Paludibacter, Spirochaeta, Geovibrio and/or Azovibrio. In further cases a microbial population mixture comprises archaea from the Methanobacteriaceae family, such as archaea from the methanobacterium and/or methanobrevibacter genus; Methanococcus maripaludis, alone or in combination with Desulfobacterium corrodens, etc.

A wide range of materials can be used as the material for a cathode and/or anode. For example, the cathode and/or anode can comprise carbon paper, carbon cloth, carbon felt, carbon wool, carbon foam, graphite, porous graphite, graphite powder, graphene, carbon nanotubes, electrospun carbon fibers, a conductive polymer, platinum, palladium, titanium, gold, silver, nickel, copper, tin, iron, cobalt, tungsten, stainless steel, and combinations thereof. Thus, in certain aspects, the cathode and/or anode is a graphite cathode and/or anode, such as a graphite granule cathode and/or anode. In yet further aspects the cathode and/or anode is a steel cathode and/or anode, or derived from other metals.

The average cathode voltage potential can be between −300 to −900 mV, −400 to −800 mV, −400 to −600 mV, etc. In some embodiments, the microbial culture is maintained in the presence of constant cathode voltage potential. In some embodiments the cathode voltage potential is applied intermittently.

In some embodiments the culture medium is a minimal medium, e.g. free of organic compounds at the initiation of culture. In some embodiments the anodic medium comprises biowaste, including without limitation brewery waste, sewage waste, landfill, and the like.

In some embodiments culture medium in the bioreactor is exchanged periodically. In some embodiments the bioreactor is supplied with a continuous in flow of fresh media. In some embodiments the bioreactor medium is maintained without exchange for an extended period of time, e.g. for up to about 10 days, for up to about 20 days, for up to about 30 days, for up to about 40 days, for up to about 50 days, for up to about 75 days, for up to about 100 days or more.

A number of configurations can be used for the methane storage unit, which can be configured in a unit with the bioreactor, or may be remote, e.g. converted to liquid natural gas or a natural gas grid. For example, the storage unit may use a conventional storage configuration, where methane is stored as compressed natural gas (CNG) at 207 bar in pressure vessels utilizing a compression stage. As an alternative, adsorbed natural gas is stored as an adsorbed phase in a porous solid at a lower pressure, e.g. at greater than about 50 bar, greater than about 100 bar, greater than about 150 bar as 150 v/v. Adsorbents with high methane capacity include, for example, activated carbons; zeolites, metal-organic frameworks, and crystalline materials formed from metal-oxide clusters bridged by functionalized organic links and known as isoreticular metal-organic frameworks (IRMOFs).

The methane combustion element performs methane oxidation, which reacts methane with oxygen, e.g. in a fuel cell or via combustion. In some embodiments the element is an oxyfuel reactor that produces electricity and a water/CO2 effluent, from which CO2 is readily recycled for use in the bioreactor. Oxy-fuel cycles are based on the close-to-stoichiometric combustion, where the fuel is burned with enriched oxygen, for example produced in an air separation unit, and recycled flue gas. The combustion is accomplished in absence of the large amounts of nitrogen that air would have brought in if enriched oxygen was not used, and produces only CO2 and H2O. CO2 separation is accomplished by condensing water from the flue gas. Systems of interest include, without limitation, the MATIANT cycle, COOPERATE cycle, or COOLENERG cycle. See, for example, Corradetti et al. (2005) ASME J Eng Gas Turbines Power 127:545-52; Chiesa et al. (2000) ASME J Eng Gas Turbines Power 122:429-36; Desideri et al. (1999) Energy Conyers Manage 40:1899-915; Mathieu et al. (1999) J Eng Gas Turbines Power 121:116-20; Yantovski et al. (1996) Energy Conyers Manage 37(6-8):861-4; Staicovici (2002) Energy 27:831-44; Zhang et al. (2006) Energy 31:1666-79; Zhang et al. (2006) Turbine Power 128:81-91, each herein specifically incorporated by reference.

Catalytic combustion is an environmentally friendly technique for heat and power generation from methane, allowing for efficient combustion at methane gas concentrations outside of flammability limits and at temperatures lower than those used in flame combustion without undesired byproducts such as unburned hydrocarbons (UHC), carbon monoxide and oxides of nitrogen. Various membranes can be used in this element, e.g. disk-pellet membranes, hollow fiber ceramic membranes including asymmetric membranes; and the like. The element may comprise means of pumping oxygen as a driving force for the reaction.

An oxy-fuel natural gas turbine system with integrated steam reforming and CO₂ capture and separation may be used as the third element. The steam reforming heat is obtained from the available turbine exhaust heat, and the produced syngas is used as fuel with oxygen as the oxidizer. Internal combustion is used, which allows a very high heat input temperature. Moreover, the turbine working fluid can expand down to a vacuum, producing an overall high-pressure ratio.

What is claimed is:

1. An integrated system for energy conversion and storage, comprising:
a first element microbial bioreactor, operably joined to a second element methane storage unit, operably joined to third element methane fuel cell or combustion unit, where CO2 produced is recycled to the first element.

2. The system of claim 1, wherein the microbial bioreactor performs electromethanogenesis.

3. The system of claim 2, wherein the electricity for electromethanogenesis is derived from solar, hydroelectric or wind power linked to the bioreactor.

4. The system of claim 1, wherein the second element stores methane as compressed natural gas.

5. The system of claim 1, wherein the second element stores methane as adsorbed natural gas.

6. The system of claim 1, wherein the third element is an oxyfuel cell.

7. The system of claim 1, wherein the third element is an oxy-fuel natural gas turbine system with integrated steam reforming and CO₂ capture and separation.

8. A method for energy conversion and storage, comprising inputting electric power into the system of claim 1, and utilizing the output electricity.
